(19) United States
(12) Plant Patent Application Publication
(10) Pub. No.: US 2013/0276180 P1
(43) Pub. Date: Oct. 17, 2013

(54) Pummelo tree named '5-1-99-5'

(50) ;

(71) Applicant: Jude W. Grosser, Winter Haven, FL (US)

(72) Inventor: Jude W. Grosser, Winter Haven, FL (US)

(73) Assignee: Florida Foundation Seed Producers, Inc., Marianna, FL (US)

(21) Appl. No.: 13/506,364

(22) Filed: Apr. 13, 2012

(51) Int. Cl. A01H 5/00

(52) U.S. Cl. PLT/201

(57) ABSTRACT

A new red pummelo tree particularly distinguished by producing delicious and juicy red-fleshed fruit in central Florida, is disclosed.

### LATIN NAME OF THE GENUS AND SPECIES OF THE PLANT CLAIMED

Citrus grandis (L.) Osbeck

### VARIETY DENOMINATION

‘5-1-99-5’

### BACKGROUND OF THE INVENTION

The present invention relates to a new and distinct cultivar of pummelo tree, botanically known as Citrus grandis, and hereinafter referred to by the name ‘5-1-99-5’. Cultivar ‘5-1-99-5’ is a red pummelo selected from a group of open-pollinated seedlings obtained from the ‘Hirado Buntan Pink’ pummelo, currently the most popular variety in Florida. ‘5-1-99-5’ was asexually propagated by standard inverted-T grafting to sour orange+Carrizo somatic hybrid rootstock and planted in Lake Alfred, Fla., where trees demonstrated all the characteristics of the original plant.

### SUMMARY OF THE INVENTION

The following are the most outstanding and distinguishing characteristics of this new cultivar when grown under normal horticultural practices in Florida. ‘5-1-99-5’ is a medium-large sized pummelo with thinner rind than most favored pummelo selections. ‘5-1-99-5’ features attractive dark red-fleshed segments that are more uniform in size and shape than other pummelos. ‘5-1-99-5’ has a delicious flavor with just a hint of grapefruit bitterness. ‘5-1-99-5’ fruit is quite juicy and fruit size is very uniform. Fruit on the tree sometimes exist in clusters, and the rind can exhibit pink blushes where fruit are touching.

### BRIEF DESCRIPTION OF THE DRAWINGS

This new red pummelo plant is illustrated by the accompanying photographs which show the plant's form, foliage, and fruit. The colors shown are as true as can be reasonably obtained by conventional photographic procedures. All photographs were taken in November of 2010.

FIG. 1. Shows whole and cut fruit of ‘5-1-99-5’.

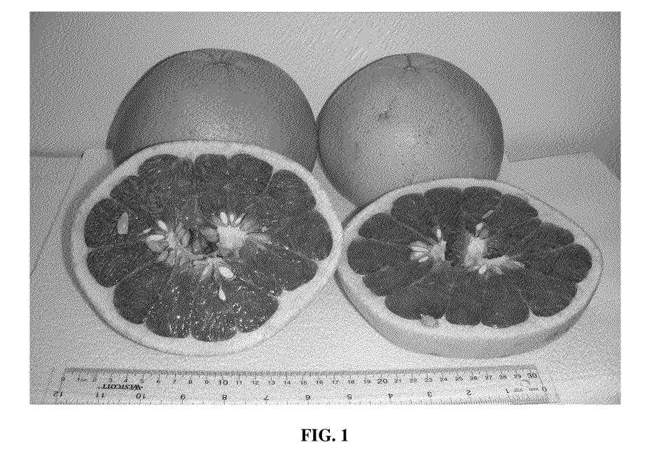

FIG. 2. Shows a tree showing overall plant habit, including foliage and fruit.

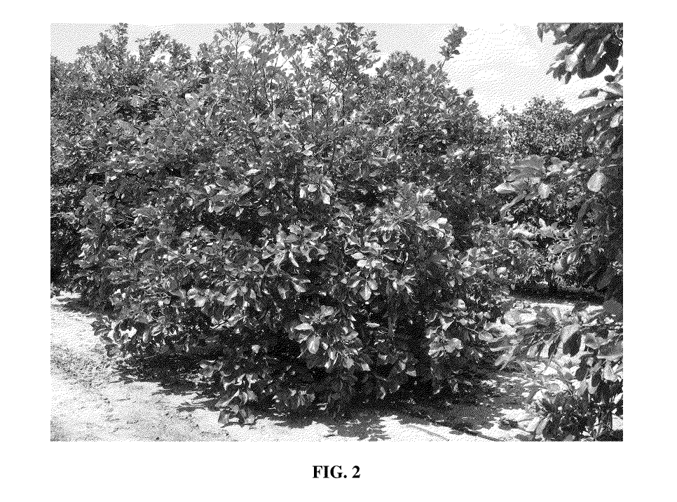

FIG. 3. Shows a cluster of fruit on a ‘5-1-99-5’ tree.

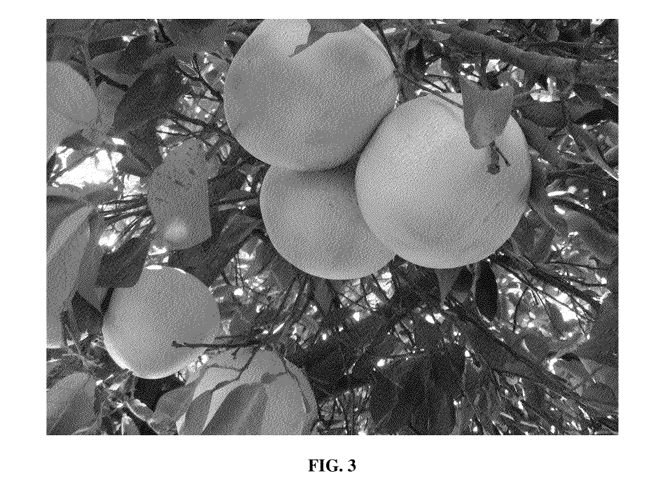

FIG. 4. Shows mature leaves and immature fruit.

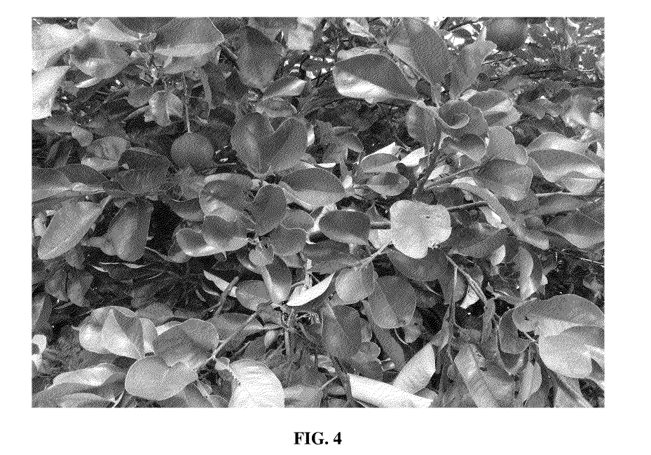

FIG. 5. Shows a fruit of ‘5-1-99-5’.

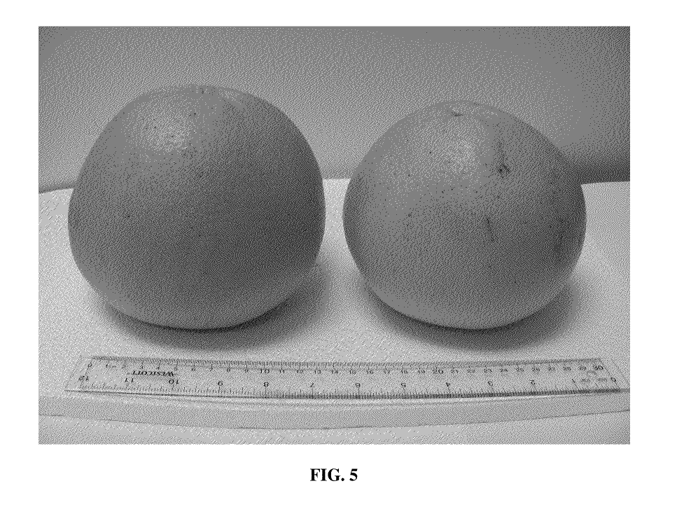

### DETAILED BOTANICAL DESCRIPTION

The following detailed description sets forth the distinctive characteristics of ‘5-1-99-5’. The present botanical description is that of the variety grown on an 8-year-old tree on Swingle citrumelo rootstock (C. paradisi×Poncirus trifoliata) in Lake Alfred, Fla. The colors (except those in common terms) are described from R.H.S. Colour Chart published by the Royal Horticultural Society in London (second edition), in association with the Flower Council of Holland.
Phenotypic Description of Citrus grandis (L.) Osbeck (‘5-1-99-5’).
Classification:
   Botanical.—Citrus grandis (L.) Osbeck.
   Common name.—Pummelo.
Parentage:
   Female parent.—‘Hirado Buntan Pink’ pummelo.
   Male parent.—Unknown.
Tree:
   Ploidy.—Diploid.
   Size.—Large, with heights of 3.35 meters currently un-pruned.
   Tree spread.—Tree spread reaches 3.4 meters.
   Vigor.—vigorous, growing shoots of 0.6 meters or more in typical new leaf flush from the current tree.
   Density.—Canopies are quite dense with open canopy.
   Form.—The shape of the tree is obloid with upright branches grown, branch growth toward medium angles. Branches exhibit drooping after fruit has commenced.
   Growth habit (current season).—Branches grow laterally, and more branches droop as fruits grow bigger.
Trunk and branches:
   Trunk diameter.—Trunk diameter reaches 13 cm at 30 cm above the ground.
   Trunk texture.—Smooth.
   Trunk bark color.—RHS 2197A (greyed-green); irregularly striated with N189A (greyed-green) and RHS N189C (greyed-green).
   Branch length.—branch length reach 3.5 meters from the first crotch point to the tip of the branch.
   Branch texture.—Relatively smooth with small thorns or spines at each leaf knot.
   Branch color (shoots from previous flush, hardened and 4 mm to 5 mm in diameter).—RHS 1138A (green).
   Crotch angle.—Tree crotch forms 30 degree angle from the main trunk.
Leaves:
   Size (lamina average).—Length: 120 mm. Width: 76 mm. L/W ratio: 1.5 to 1.6.
   Thickness.—Regular and average for commercial pummelo.
   Type.—Simple.
   Shape.—Elliptical.
   Apex.—Retuse.
   Base.—Acute to sub-obtuse.
   Margin.—Entire, smooth and very slightly undulated.
   Surface.—Upper surface: Glabrous. Lower surface: Large veins that are pinnately netted.
   Color.—Upper surface (adaxial): RHS 138A (green). Lower surface (abaxial): RHS 144b (yellow-green).
   Petiole.—Shape: Brevipetiolate (shorter than leaf lamina); junction between petiole and lamina is articulate. Width (petiole wing): Wide, compared to sweet oranges and mandarins. Shape (petiole wing): Obovate. Length: 40mm to 45 mm. Width: 20 mm to 28 mm. Color: RHS 138A (green).
Flowers:
   Type.—Hermaphrodite.
   Bearing.—Flower clusters grow from leaf axillaries and branch terminals, each flower branch consisting of 2-5 flower clusters, one cluster normally consisting of 7-10 flowers.
   Flower born.—Flower branch derived from the stems of flush of previous year.
   Diameter.—Fully opened flowers average 43 mm to 52 mm.
   Depth.—Typical flower depth averages between 22 mm to 26 mm.
   Flower bud.—Initial visible flower bud shape: Round dome shaped. Initial visible flower bud length: 3-4 mm. Initial visible flower bud diameter: 3 mm. Mature flower bud shape (before opening): enlongated olive shape. Mature flower bud length (before opening): 18 mm to 19 mm. Mature flower bud diameter (before opening): 12.5 mm-13 mm.
   Color.—RHSNN145A (yellow-green) for initial visible flower bud.
   Petals.—Number: 5. Shape: Flat spatula shaped. Apex shape: Smooth acute shaped. Base shape: Even Obtuse. Color: Upper surface: RHS 155B (white). Lower surface: RHSNN155C (white) with yellow-green (RHS 150A) spots distributed in the middle section of the petal toward to the petal apex. Margin: Smooth. Length: 22 mm. Width: 8 mm.
   Calyx.—Size: Large. Length: 13.6 mm. Diameter: 7.8 mm to 8.2 mm.
   Sepal.—Number: 4 per flower. Shape: Short flat delta shaped with short acute angle at the apex. Length: 4.9 mm. Width: 7.6 mm. Apex shape: Acute triangle shape. Margin: Smooth. Color: RHS 145C (yellow-green).
   Pedicel.—Length: 13.7 mm to 15.4 mm. Diameter: 1.9 mm to 2.5 mm. Color: RHS 142C (green).
   Flower blooming period.—First bloom: around late March to early April in central Florida. Full bloom: In 2010, observed full bloom in mid-April.
   Fragrance.—Very fragrant compared to other citrus blossoms.
Reproductive organs:
   Fertility.—Self-fertile.
   Stamen.—Number: 25-27. Length: 12.8 mm to 14.2 mm.
   Pistil.—Number: 1. Color: RHS 142D (green). Length: 12 mm. Diameter: 2.0 mm to 2.7 mm.
   Stigma color.—RHS 154C (yellow-green).
   Style.—Length: 8.5 mm. Diameter: 2.0 mm to 2.4 mm. Color: RHS 142D (green).
   Ovary.—Shape: Oval shaped. Diameter: 3.8 mm to 5 mm. Color: RHS 144D (yellow-green).
   Anther.—Length: 1.6 mm. Width: 0.65 mm. Color: RHS 13C (yellow).
   Anther filament length.—8.3 mm to 10.3 mm.
   Pollen amount.—Abundant.
   Pollen color (general): RHS 12A (bright-yellow).
Fruit:
   Size.—Uniform (more so than most other pummelo cultivars), 160 mm to 180 mm in diameter, 140 mm to 160 mm in tall.
   Fruit weight.—averages 1,700 grams.
   Shape.—Round and slightly bell shaped.
   Shape (cross-section).—Nearly round.
   Apex.—Truncated with shallow dent mark.
   Apex cavity diameter.—None.
   Base.—No neck, with slightly rough peel surface.
   Base cavity diameter.—9.7 mm.
Harvest: Mid-October through January (affected by rootstock).
Fruit stem (short stem connecting fruit to the branch):
   Length.—24 mm.
   Diameter.—7.5 mm.
   Color.—RHS 188A (grey-green) with RHS 199D (grey-brown) strip.
Rind:
   Adherence.—Albedo (mesocarp) to fresh (endocarp) is strong compared to mandarins, but similar to grapefruit.
   Thickness.—8 mm to 10 mm, thinner than most other common pummelo varieties.
   Texture.—Medium firm, softer when fully ripe.
   Color.—Flavedo (epicarp): Range from between RHS 1C (green-yellow) to RHS 2B (yellow). Albedo (mesocarp): RHS 36D (red) to RHS 36A (red). Stylar end: Closed.
Rind oil cell density: 70 to 84 oil cells per square centimeter.
Flesh:
   Number of segments.—Average between 12 and 13 segments per fruit.
   Segment walls.—Strong compare to grapefruit varieties with sufficient strength to maintain integrity as separated.
   Juice.—Abundant.
   Color.—Uniformly RHS 39A (red).
   Texture.—Medium-soft and able to maintain flesh integrity as separated from segment wall.
   Vesicles.—Medium-thick. Length: averages 18 mm to 26 mm. Diameter: averages 3.8 mm to 7 mm.
Flavor and quality: Excellent flavor from mid-October through January (data from Jan. 4, 2010).
   Acidity (average).—1.1.
   Sucrose content.—11.0.
   Brix/acid ratio.—10.
   Color score.—43.2-45.0 (slightly variable per fruit).
Seeds:
   Type.—Monoembryonic.
   Number.—Ranges from 120 to 130.
   Shape.—Various in Clavate/club shaped to deltoid-shaped.
   Size.—Length: 15 mm to 18 mm. Width: 7 mm to 9 mm.
   Seed coat color.—Outer Surface: RHS 158A (yellow-white) and slightly wrinkled. Inner surface: RHS 164B (greyed-orange).
   Cotyledon color.—RHS NN155A (white).

What is claimed is:

1. A new and distinct red pummelo tree as shown and described herein.
